(19) United States
(12) Patent Application Publication
(10) Pub. No.: US 2022/0099679 A1
(43) Pub. Date: Mar. 31, 2022

(54) Portable diagnostic device for viewing biological entities and structures

(71) Applicants: Sylvester Tumusiime, Metairie, LA (US); Jemma Taylor, Ashford (GB)

(72) Inventors: Sylvester Tumusiime, Metairie, LA (US); Jemma Taylor, Ashford (GB)

(21) Appl. No.: 17/033,827

(22) Filed: Sep. 27, 2020

(51) Int. Cl. G01N 33/58; G01N 21/64

(52) U.S. Cl. CPC G01N 33/582; G01N 21/6428; G01N 2021/6478; G01N 2021/6439; G01N 21/645

(57) ABSTRACT

The present invention seeks to provide an easily portable means of visualizing biological samples by way of fluorescent emissions from fluorescently tagged antibodies associated with the biological entities; thereby acting as a diagnostic tool when appropriate antibodies and samples are applied to the sample plate. Current methods used to visualize biological entities by way of fluorescent emissions from fluorescently tagged antibodies associated with the biological samples involve the use of large bulky equipment that doesn't exist in a modular format—different components existing as disparate disjointed units that cannot be physically associated or linked with each other. This invention significantly decreases the size of the components needed to visualize biological samples by way of fluorescent emissions from fluorescently tagged antibodies and also modularizes the components such that they can be connected to each other to form the portable detection device.

### CROSS-REFERENCE TO RELATED APPLICATIONS

Not applicable.

### STATEMENT REGARDING FEDERALLY SPONSORED RESEARCH OR DEVELOPMENT

This invention was not funded by federal research funds.

### REFERENCE TO SEQUENCE LISTING, A TABLE, OR A COMPUTER PROGRAM LISTING COMPACT DISC APPENDIX

Not applicable.

### BACKGROUND OF THE INVENTION

Coronaviruses are single-stranded RNA viruses that belong to the subfamily Coronavirinae of the family Coronaviridae and the Nidovirales family¹ (Non Patent Literature 1). They mainly affect the respiratory and gastrointestinal systems of the organisms that they infect. Up to 7 different Coronaviruses have been identified. These include: HCoV-NL63, HCoV-229E, HCoV-0C43, HCoVHKU1, SARS-CoV, MERS-CoV and SARS-CoV-2² (Non Patent Literature 2).

The SARS-CoV-2 genome encodes up to 27 proteins including the spike surface glycoprotein protein (S), small envelope protein (E), matrix protein (M) and nucleocapsid protein (N)³ (Non Patent Literature 3). The S-protein has two domains with distinct functions: the 51 domain which is responsible for angiotensin-converting enzyme 2 (ACE2) receptor binding and the S2 domain which is responsible for cell membrane fusion⁴ (Non Patent Literature 4). While there exist regions of homology between S-proteins belonging to SARS-CoV and SARS-CoV-2⁵ (Non Patent Literature 5), the S-protein exhibits high variability of amino acid sequences in coronavirus⁶ (Non Patent Literature 6). Specifically, one region of significant heterogeneity between SARS coronavirus Tor2, SARS coronavirus GD01 and SARS-CoV-2 occurs between amino acid residues 442 and 472 of the spike proteins (Non Patent Literature 5). In this region, the SARS-CoV-2 protein sequence is markedly different from that of the other SARS coronaviruses. Furthermore, the 51 domain (residues 318-510) of the SARS-CoV S-protein has been shown to be a potent candidate for antibody generation⁴ (Non Patent Literature 4).

Because Coronaviruses are highly transmittable, there is a need to rapidly diagnose individuals that are positive for the virus so as to establish quarantine measures that limit movement leading to decreased human to human transmission. Diagnosis of patient infection by the SARS-CoV coronaviruses has been achieved mainly via clinical characteristics, epidemiological history, chest imaging and laboratory detection (positive result from high-throughput sequence, an RT-PCR assay, the presence of anti-SARS-COV IgM/IgG antibodies and enzyme-linked immunosorbent assay (ELISA)). RT-PCR tests have been developed for the RNA-dependent RNA polymerase (RdRp) gene of the ORF1ab sequence, E gene, N gene, and S-gene of SARS-CoV-2⁷⁻¹⁰ (Non Patent Literature 7-10)—nucleic acid detection observable in nasal and pharyngeal swabs, bronchoalveolar lavage fluid, sputum, bronchial aspirates, blood, and anal swabs^{2,11} (Non Patent Literature 2, 11). Field-deployable, rapid diagnostic tools based on saliva samples assayed for copies of the coronavirus have been developed¹² (Non Patent Literature 12)—largely due to the ease of collection and the reduced risk to health workers involved in the collection¹³ (Non Patent Literature 13). In addition, point-of-care, rapid-diagnostic tools including: the Xpert Xpress SARS-CoV-2 test¹⁴ (Non Patent Literature 14), a self-contained cartridge system that provides results in 45 minutes; the Accula SARS-CoV-2 handheld device and, the ID NOW COVID-19¹⁵ (Non Patent Literature 15) have been developed. These tools are all based on PCR amplification of target nucleic acids. Other molecular-based point-of-care diagnostic tools include: loop-mediated isothermal amplification (LAMP)- and clustered regularly interspaced short palindromic repeats (CRISPR)-based methodologies^{16,17} (Non Patent Literature 16, 17).

While immunofluorescent-based methods exist to detect and visualize viral proteins expressed in Cells^{18,19} (Non Patent Literature 18, 19), no FDA-approved point-of-care diagnostic kits exist²⁰ (Non Patent Literature 20). Furthermore, none of the immunofluorescence-based methods are targeted to extracellular viral particles that exist in body fluids.

### BRIEF SUMMARY OF THE INVENTION

The invention described herein provides a portable means for the detection of viral particles and other biological entities in a sample. Furthermore, also provided herein is a detection cell comprised of a sample plate to which target part particles are attached either directly or indirectly. Also provided is a laser emission cell for exciting appropriate fluorophores associated with target viral particles or biological sample and a magnifier to amplify the fluorescent emissions. The invention is designed for use with a portable recording device such as a smart phone or other similar portable device that can capture fluorescent emissions.

Detection of a target particle (such as a viral particle or other biological particle with appropriate surface characteristics) is mediated in part by binding of the target particle to the sample plate, either directly or indirectly followed by binding of appropriate fluorescently-tagged antibody to the bound particle and excitation of the fluorescently tagged antibody with an appropriate wavelength to cause excitation and fluorescent emission.

In one embodiment of the invention, provided herein is one method of detecting a biological particle such as virus comprising the steps of a) applying the sample to the sample plate under conditions that enable attachment of the biological particle to the sample plate surface; b) applying sample buffer with appropriate fluorescently tagged antibody to the bound biological particles and allowing sufficient time for binding; c) adding a sample buffer to wash off any unbound biological particles and antibody from the surface of the sample plate; and d) exciting the fluorescent tag of bound antibodies with appropriate light wavelength and detecting the emitted light.

In yet another embodiment of the invention, provided herein is a method of detecting a biological particle such as a virus comprising the steps of a) applying sample buffer with appropriate fluorescently tagged antibody to the sample plate; b) removing excess fluorescently tagged antibody from the sample plate; c) applying the sample to the sample plate containing appropriate fluorescently tagged antibody under conditions that enable attachment of the biological particle to the fluorescently tagged antibody; d) removing excess sample from the sample plate; e) washing off any unbound biological particles from the surface of the sample plate; f) exciting the fluorescent tag with appropriate light wavelength and detecting the emitted light.

In a further embodiment of the invention, provided herein is a method of detecting a biological particle such as a virus comprising the steps of a) applying sample buffer with appropriate primary antibody to the sample plate to enable binding of the primary antibody to the sample plate; b) removing excess primary antibody from the sample plate; c) applying the sample to the sample plate containing bound primary antibody under conditions that enable attachment of the biological particle to the bound primary antibody; d) removing excess sample from the sample plate; f) washing off any unbound biological particles from the surface of the sample plate containing the bound primary antibody; g) applying sample buffer with appropriate secondary fluorescently tagged antibody to the sample plate containing bound biological particles to allow for binding of the secondary antibody to biological particles; h) washing off excess unbound secondary fluorescently tagged antibody; i) exciting the fluorescent tag with appropriate light wavelength and detecting the emitted light.

### BRIEF DESCRIPTION OF THE SEVERAL VIEWS OF THE DRAWING

FIG. 1 is a side view of the device showing excitation of the fluorophore bound to the antibody and fluorescent emission following excitation.

FIG. 2 is the top view of the excitation-wavelength-source showing the arrangement of the excitation-wavelength-source light emitting diodes.

FIG. 3 is a longitudinal cross-section of the excitation-wavelength-source support structure showing the arrangement of the inner and outer walls of the excitation-wavelength-source support structure and the transmission channel for the electromagnetic energy released by the excitation-wavelength-source light emitting diode.

### DETAILED DESCRIPTION OF THE INVENTION

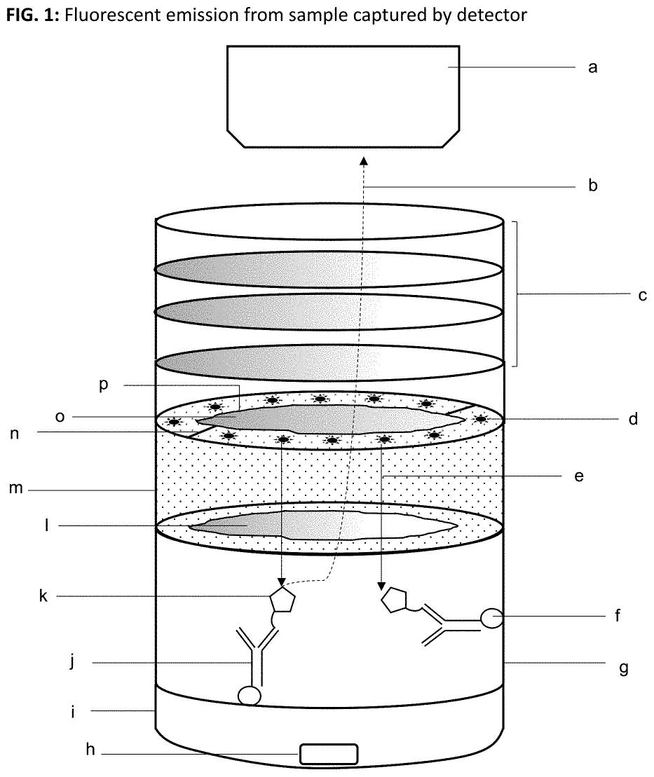

FIG. 1a Is a side view of the detection device. The detection device will have the capacity to capture emitted excitation wavelengths from the fluorescent tag (FIG. 1k) bound to antibody (FIG. 1j) associated with the target of interest. Such detection device will also have the capacity to record and store captured images of the emitted excitation wavelengths.

FIG. 1b shows a representation of a fluorescent emission from an excited fluorophore (FIG. 1k). The fluorescent emission will result from the absorption of electromagnetic energy directed from an excitation-wavelength-source light emitting diode (FIG. 1d).

FIG. 1c shows a representation of the magnifying-plane stack. The magnifying-plane stack amplifies and focuses the emitted excitation wavelength (FIG. 1b) emitted by an excited fluorophore (FIG. 1k).

FIG. 1d shows one excitation-wavelength-source light emitting diode. The excitation-wavelength-source light emitting diodes are contained in the excitation-wavelength source support structure (FIG. 1m) and they act to release electromagnetic energy at a specific wavelength tuned to excite a given fluorophore.

FIG. 1e shows excitation wavelength travelling to target fluorophore.

FIG. 1f shows biological entity bound to sample plate wall. The biological entity possesses an antigen that is recognizable by the fluorescently tagged antibody (FIG. 1j).

FIG. 1g shows the exterior wall of the sample plate.

FIG. 1h shows the power source for the excitation-wavelength-source light emitting diode.

FIG. 1i shows the exterior wall of the enclosure of the power source for the excitation-wavelength-source light emitting diode.

FIG. 1j shows primary antibody with fluorescent tag attached to target surface. Attachment of the primary antibody to the target surface is dependent on recognition of a specific epitope.

FIG. 1k shows fluorophore emitting fluorescence following excitation by excitation wavelength.

FIGS. 1l and 1o show the lower and upper portals respectively of the excitation-wavelength-source support structure through which fluorescent emissions from the fluorophore (FIG. 1k) traverse prior to capture by the detection device (FIG. 1a). The lower and upper excitation portals are created by the inner wall (FIG. 1p) of the excitation-wavelength-source support structure and they minimize contamination of the fluorescent emission (FIG. 1b) from the fluorescent tag with excitation wavelength released by the excitation-wavelength-source light emitting diode (FIG. 1d).

FIG. 1m shows the excitation-wavelength-source exterior wall support structure.

FIG. 1n shows the excitation-wavelength-source support structure inter-wall connector.

FIG. 1p shows the inner wall of the excitation-wavelength-source support structure.

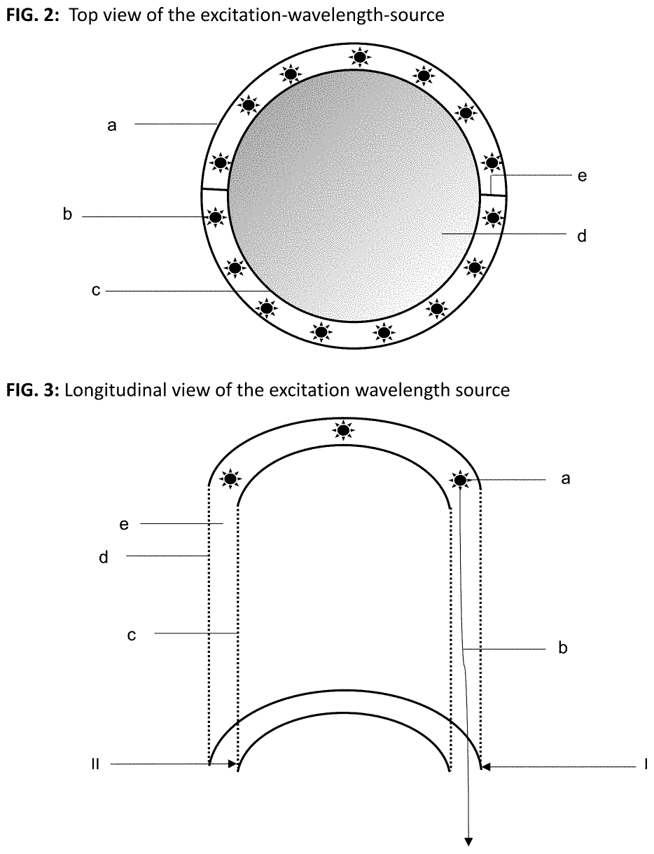

FIG. 2a shows a top view of the exterior wall of the excitation-wavelength-source support structure.

FIG. 2b shows a top view of the excitation-wavelength-source light emitting diode.

FIG. 2c shows a top view of the inner wall of the excitation-wavelength-source support structure.

FIG. 2d shows a top view of the upper portal of the excitation-wavelength-source support structure.

FIG. 2e. shows a top view of the excitation-wavelength-source support structure inter-wall connector.

FIG. 3a shows a longitudinal view of the excitation-wavelength-source light emitting diode.

FIG. 3b. is a representation of an excitation wavelength from the excitation-wavelength-source light emitting diode traversing the transmission channel of the excitation-wavelength-source support structure.

FIG. 3c. shows a longitudinal view of the inner wall of the excitation-wavelength-source support structure.

FIG. 3d. shows a longitudinal view of the outer wall of the excitation-wavelength-source support structure.

FIG. 3e. shows a longitudinal view of the transmission channel of the excitation-wavelength-source support structure.

FIG. 3I shows the point of contact between the vertical section of the exterior wall of the excitation-wavelength-source support structure and the lower circular portion of the exterior wall of the excitation-wavelength-source support structure.

FIG. 3II shows the point of contact between the vertical section of the interior wall of the excitation-wavelength-source support structure and the lower circular portion of the interior wall of the excitation-wavelength-source support structure.

### BIBLIOGRAPHY

1. Weiss, S. R. & Leibowitz, J. L. Coronavirus Pathogenesis. in Advances in Virus Research vol. 81 85-164 (Elsevier, 2011).
2. Wang, H. et al. The genetic sequence, origin, and diagnosis of SARS-CoV-2. Eur J Clin Microbiol Infect Dis 39, 1629-1635 (2020).
3. Wu, A. et al. Genome Composition and Divergence of the Novel Coronavirus (2019-nCoV) Originating in China. Cell Host & Microbe 27, 325-328 (2020).
4. He, Y. et al. Receptor-binding domain of SARS-CoV spike protein induces highly potent neutralizing antibodies: implication for developing subunit vaccine. Biochemical and Biophysical Research Communications 324, 773-781 (2004).
5. Xu, X. et al. Evolution of the novel coronavirus from the ongoing Wuhan outbreak and modeling of its spike protein for risk of human transmission. Sci. China Life Sci. 63, 457-460 (2020).
6. Hu, B. et al. Discovery of a rich gene pool of bat SARS-related coronaviruses provides new insights into the origin of SARS coronavirus. PLoS Pathog 13, e1006698 (2017).
7. Konrad, R. et al. Rapid establishment of laboratory diagnostics for the novel coronavirus SARS-CoV-2 in Bavaria, Germany, February 2020. Eurosurveillance 25, (2020).
8. Corman, V. M. et al. Detection of 2019 novel coronavirus (2019-nCoV) by real-time RT-PCR. Eurosurveillance 25, (2020).
9. Chan, J. F.-W. et al. Improved Molecular Diagnosis of COVID-19 by the Novel, Highly Sensitive and Specific COVID-19-RdRp/Hel Real-Time Reverse Transcription-PCR Assay Validated In Vitro and with Clinical Specimens. J Clin Microbiol 58, e00310-20, /jcm/58/5/JCM.00310-20.atom (2020).
10. Reusken, C. B. E. M. et al. Laboratory readiness and response for novel coronavirus (2019-nCoV) in expert laboratories in 30 EU/EEA countries, January 2020. Eurosurveillance 25, (2020).
11. Pan, Y., Zhang, D., Yang, P., Poon, L. L. M. & Wang, Q. Viral load of SARS-CoV-2 in clinical samples. The Lancet Infectious Diseases 20, 411-412 (2020).
12. Wei, S. et al. Field-deployable, rapid diagnostic testing of saliva samples for SARS-CoV-2. http://medrxiv.org/lookup/doi/10.1101/2020.06.13.20129841 (2020) doi:10.1101/2020.06.13.20129841.
13. Harikrishnan, P. Saliva as a Potential Diagnostic Specimen for COVID-19 Testing. J Craniofac Surg (2020) doi:10.1097/SCS.0000000000006724.
14. Loeffelholz, M. J. et al. Multicenter Evaluation of the Cepheid Xpert Xpress SARS-CoV-2 Test. J Clin Microbiol 58, e00926-20, /jcm/58/8/JCM.00926-20.atom (2020).
15. Chau, C. H., Strope, J. D. & Figg, W. D. COVID-19 Clinical Diagnostics and Testing Technology. Pharmacotherapy phar.2439 (2020) doi:10.1002/phar.2439.
16. Baek, Y. H. et al. Development of a reverse transcription-loop-mediated isothermal amplification as a rapid early-detection method for novel SARS-CoV-2. Emerging Microbes & Infections 9, 998-1007 (2020).
17. Broughton, J. P. et al. CRISPR-Cas12-based detection of SARS-CoV-2. Nat Biotechnol 38, 870-874 (2020).
18. Sandstrom, E. et al. Detection of human anti-HTLV-III antibodies by indirect immunofluorescence using fixed cells. Transfusion 25, 308-312 (1985).
19. Hedenskog, M. et al. Testing for antibodies to AIDS-associated retrovirus (HTLV-III/LAV) by indirect fixed cell immunofluorescence: Specificity, sensitivity, and applications. J. Med. Virol. 19, 325-334 (1986).
20. Simonetti, R. F., Dewar, R. & Maldarelli, F. Diagnosis of Human Immunodeficiency Virus Infection. in Principles and Practive of Infectious Diseases vol. 1 (Elsevier, 2015).

What is claimed is:

1. A method of visualizing biological entities or structures using a single portable device, comprising: (a) directly or indirectly applying a biological sample that is directly or indirectly associated with a fluorescently-tagged antibody onto a sample plate; (b) physically connecting the sample plate with contained fluorescently-tagged biological sample to an excitation-wavelength-source support structure that holds light emitting diodes that emit excitation wavelengths of appropriate wavelength targeted at the fluorescently-tagged antibody associated with the biological sample (light emitting diodes being powered by a power source connected to the portable device) such that the sample plate and excitation-wavelength-source support structure form one continuous object; (c) amplifying and focusing the emitted excitation wavelength from the fluorescently-tagged antibody associated with the biological entity towards a detection device using a stack of appropriate lenses or single lens physically connected to the sample plate and excitation-wavelength-source support structure so that the sample plate, excitation-wavelength-source support structure and lens(es) form a single modular structure.
